(19) United States
(12) Patent Application Publication
(10) Pub. No.: US 2006/0252684 A1
(43) Pub. Date: Nov. 9, 2006

(54) Osteopontin-based cancer therapies

(75) Inventor: Georg F. Weber, Cincinnati, OH (US)

Correspondence Address: FISH & RICHARDSON PC, P.O. BOX 1022, MINNEAPOLIS, MN, 55440-1022, US

(73) Assignee: New England Medical Center Hospitals, Inc., a Massachusetts corporation

(21) Appl. No.: 11/272,528

(22) Filed: Nov. 10, 2005

Related U.S. Application Data
(63) Continuation of application No. 10/678,355, filed on Oct. 2, 2003, now abandoned.
(60) Provisional application No. 60/415,712, filed on Oct. 2, 2002.

(51) Int. Cl. A61K 48/00; A61K 38/54

(52) U.S. Cl. 514/12; 514/44

(57) ABSTRACT

The invention relates to therapies for treating cancer patients by targeting the osteopontin isoforms OPN-b and OPN-c. Osteopontin is a cytokine that is essential for cellular immunity, particularly through its full length form, OPN-a. OPN-b and OPN-c are splice variants that lack exons 5 and 4, respectively, of the protein's six translated exons. The invention provides methods for treating cancer patients with therapeutics that inhibit or degrade the OPN-b or OPN-c isoforms specifically, thereby leaving the innocuous OPN-a form intact and available to perform its normal functions in the cell.

### RELATED APPLICATIONS

This application claims the benefit of U.S. Provisional Application No. 60/415,712, filed Oct. 2, 2002, which is incorporated herein by reference in its entirety.

### GOVERNMENT SUPPORT

The work described herein was carried out, at least in part, using funds from the U.S. government under grant number DAMD 17-98-1-806, awarded by the U.S. Army Medical Research and Material Command (USAMRMC), and grant number CA76176, awarded by the National Cancer Institute (NCI). The government may therefore have certain rights in the invention.

### FIELD OF THE INVENTION

The present invention relates to methods of identifying malignancies and treating cancer patients by identifying and targeting the osteopontin (OPN) isoforms OPN-b and OPN-c.

### BACKGROUND

Osteopontin is a cytokine that has been associated with a variety of physiological processes. For example, it supports host cell resistance by inducing immune cells to migrate and invade sites of inflammation; it promotes neovascularization; it inhibits apoptosis (Reviewed by Weber, Biochim Biophys Acta 1552:61-85, 2001); and it can confer metastatic behavior in a variety of cell types. With respect to metastases, there is some indication that tumor cells produce forms of osteopontin that are structurally and functionally distinct from those produced by untransformed cells. For instance, cells within an osteosarcoma produce a smaller form of osteopontin than do normal bone cells (Kasugai et al., Bone Miner. 13:235-250, 1991), and malignant cells often secrete hypophosphorylated osteopontin variants (Shanmugam et al., Biochem 36:5729-5738) or a splice variant that contains a N-terminal deletion (Kiefer et al., Nuc Acids Res 17:3306, 1989). In addition, tumor-derived osteopontin is unable to associate with the extracellular matrix (Rittling et al., J. Biol. Chem. 277:9175-9182, 2002).

There are several osteopontin splice variants. The osteopontin mRNA transcript includes seven exons, six of which are translated (exon 1 is not translated), and three splice variants have been identified: osteopontin-a (OPN-a) mRNA contains all seven exons, osteopontin-b (OPN-b) mRNA lacks exon 5, and osteopontin-c (OPN-c) mRNA lacks exon 4 (Saitoh et al., Lab. Invest. 72:55-63, 1995). Integrin binding sites are located in a central part of the protein and are primarily encoded by exon 6. The osteopontin receptor, CD44, binds the C-terminus.

### SUMMARY

The present invention is based, in part, on the discovery that two specific RNA splice variants of osteopontin, OPN-b and OPN-c, are expressed in a variety of tumor cell lines, but not in normal tissues (e.g., non-cancerous tissue) or in benign tumors. Accordingly, the invention features, inter alia, methods for treating a patient who has a cancer associated with OPN-b and/or OPN-c expression; methods for determining whether a patient has a malignant, rather than a benign, growth; and methods for detecting or identifying agents that inhibit the expression or activity of OPN-b or OPN-c.

The methods of treating or preventing cancer can be carried out by inhibiting the expression of OPN-b and/or OPN-c or the activity of the protein it encodes (sequences and SEQ ID NOs. are provided below). To inhibit the expression of OPN-b or OPN-c, one can administer one or more inhibitory agents, such as an antisense RNA sequence, a small inhibitory RNA (siRNA), or a ribozyme, any of which can be designed to target a sequence within OPN-b or OPN-c (and preferably exclusively within either of these isoforms). For instance, the exon 4/exon 6 splice junction would be a target for RNA- or nucleic acid-based therapies (e.g., antisense, siRNA, or ribozyme therapeutics) against OPN-b mRNA. Similarly, the exon 3/exon 5 junction would be a target of OPN-c-specific therapies. Generally, “antisense” RNA sequences are complementary to all or a part of the coding sequence of an mRNA, although there may be some “mismatch” so long as the antisense RNA hybridizes with and inhibits translation of the mRNA. siRNAs are generally short (e.g. 21-23 nucleotides long) double stranded RNA (dsRNA) containing 1-2 nucleotide 3′ overhangs. While the methods of the invention are not limited to agents that inhibit osteopontin by any particular mechanism, in the case of siRNA, it is expected that, since one strand of the dsRNA will be homologous to osteopontin-b (or osteopontin-c) mRNA, it will direct-osteopontin-b (or osteopontin-c) RNA cleavage by the RNAseIII-like enzyme Dicer within the RNA induced silencing complex (RISC). Ribozymes are structured RNAs that can catalyze chemical reactions resulting in specific breakdown of osteopontin-b and/or osteopontin-c RNAs.

Administering RNA-based therapeutics such as those described above can lead to partial or substantially complete silencing of OPN-b or OPN-c mRNA (e.g., mRNAs of the two isoforms can be degraded, inhibited, or otherwise rendered inactive to such an extent that they fail to substantially contribute to pathogenesis (e.g., cancer or tumor growth or metastases) and there is an improvement in an objective sign or clinical symptom in the patient being treated or a decrease in the risk that an OPN-b expressing cancer or an OPN-c expressing cancer will occur, grow, spread, or recur). Dosages, formulations, and routes of administering OPN-b or OPN-c inhibitors are discussed further below. The amount of any agent that inhibits OPN-b or OPN-c, whether that agent acts by inhibiting the expression or activity (agents that inhibit activity are discussed below) of these isoforms, can be a “therapeutically effective” amount (e.g., an amount sufficient to improve an objective sign or clinical symptom of the cancer in the patient being treated or when it reduces the risk that an OPN-b expressing (or OPN-c expressing) cancer will occur, grow, spread, or recur).

Alternatively, or in addition, one can administer an agent that inhibits the activity of OPN-b or OPN-c protein. Accordingly, the methods of the invention encompass administering a peptide or non-peptide agent (or one or more of each or both) to treat a patient with an OPN-b or OPN-c expressing cancer. Non-peptide agents include chemical compounds (e.g., small molecules) and antibodies. The antibodies will be immunoglobulin molecules having a specific amino acid sequence, by virtue of which they interact with the protein antigen (here, OPN-b, OPN-c, or fragments thereof) that induced the antibody's synthesis. Anti-OPN-b or anti-OPN-c antibodies administered to human patients can be “humanized” by methods known in the art. The antibodies administered can be monoclonal antibodies. Synthetic peptides are polymers of amino acid residues that can be chemically synthesized or produced by recombinant techniques (the amino acids are linked together by amide bonds formed between the carboxyl group of one amino acid and the amino group of another). The terms peptide and polypeptide are generally used in reference to amino acid polymers that are shorter than “proteins.” However, unless specifically noted below, there is no other intended distinction between peptides, polypeptides, and proteins. Small molecules are chemical compounds that affect the phenotype of a cell or organism by, for example, modulating the activity of a specific protein or nucleic acid within a cell. As with other anti-OPN-b or anti-OPN-c therapeutics, small molecules may affect a cell by directly interacting with either or both of the isoforms or by interacting with a molecule that acts upstream or downstream of the biochemical cascade that results in decreased OPN-b or OPN-c expression or activity.

Agents that inhibit OPN-b or OPN-c protein activity can be used to treat patients with OPN-b or OPN-c expressing cancers or to reduce the likelihood that a patient will develop such a cancer (as either an initial or recurring event). Preferably, agents employed in the methods of the invention specifically inhibit OPN-b or OPN-c (e.g., OPN-b or OPN-c protein), but absolute specificity is not necessarily required. An agent specifically inhibits OPN-b when it inhibits OPN-b to a greater extent than it inhibits OPN-a or OPN-c, or when the agent inhibits OPN-b but does not inhibit OPN-a or OPN-c to any detectable extent. Similarly, an agent specifically inhibits OPN-c when it inhibits OPN-c to a greater extent than it inhibits OPN-a or OPN-b, or when the agent inhibits OPN-c but does not inhibit OPN-a or OPN-b to any detectable extent. As with agents that inhibit the expression of OPN-b or OPN-c mRNA, agents that specifically bind (or otherwise inhibit the activity of) OPN-b or OPN-c protein can be used to treat patients who are at risk of developing an OPN-b or OPN-c expressing cancer (e.g., healthy patients with a family history of cancer (e.g., OPN-b or OPN-c expressing cancer) or patients who have been treated (e.g., by surgery or with chemotherapies or radiation therapies) for an OPN-b or OPN-c expressing cancer that may recur). Physicians, in consult with each other and their patients, can determine whether a given patient's risk (whether imposed by family history or personal history (e.g., expression of particular molecular markers such as BRCA-1, BRCA-2, or PSA, or certain events or circumstances, such as heavy smoking or exposure to carcinogens such as asbestos or radiation, including nuclear or light (e.g., ultraviolet) energy)) is sufficient to merit treatment with a therapeutic agent described herein.

Agents that inhibit the expression or activity of OPN-b may be referred to herein as “anti-OPN-b therapeutics,” and agents that inhibit the expression or activity of OPN-c may be referred to herein as “anti-OPN-c therapeutics.” Any of these agents can be combined with any known method of cancer treatment or prevention. For example, an anti-OPN-b therapeutic can be administered in connection with (i.e., before, during or after) a surgical procedure in which an OPN-b-associated tumor is physically removed from a patient. Similarly, an anti-OPN-b therapeutic can be administered in connection with (i.e., before, during or after) a radiation treatment or a course of chemotherapy. Anti-OPN-c therapeutics can be administered under the same circumstances as anti-OPN-b therapeutics. Anti-OPN-b and anti-OPN-c therapeutics can also be administered simultaneously under the same circumstances. As noted, patients amenable to treatment include those having an OPN-b or OPN-c expressing cancer. However, expressing or overexpressing OPN-a may have beneficial effects on any cancer. Accordingly, the methods of the invention can also be carried out by expressing or overexpressing OPN-a in a cell (by, for example, delivering to the patient a DNA construct that directs the expression of OPN-a or a therapeutically active fragment or other mutant thereof). These methods can be carried out in conjunction with those described above. That is, a patient can receive a therapeutic that inhibits the expression or activity of OPN-b and/or OPN-c together with a therapeutic that increases the expression or activity of OPN-a.

Cancerous cells exhibit a capacity for autonomous growth (i.e., an abnormal state or condition characterized by rapid cellular proliferation). Patients amenable to treatment include those with cancers of various organs or organ systems, including the lung, breast, thyroid, lymphoid, gastrointestinal, and genito-urinary tract, as well as adenocarcinomas, which include malignancies such as most colon cancers, renal-cell carcinoma, prostate cancer and/or testicular tumors, non-small cell carcinoma of the lung, cancer of the small intestine and cancer of the esophagus.

The invention also features methods of screening for agents that specifically inhibit the expression of OPN-b and OPN-c (the transcription of DNA into mRNA or the translation of mRNA into protein) or the activity of OPN-b and OPN-c protein. Candidate therapeutic agents can be evaluated in assays that reveal the level of OPN-b and OPN-c mRNA or protein expression. For example, one can expose a cell expressing OPN-b and OPN-c (be it an apparently healthy cell or a cancerous cell (suitable cells include MDA-MB435 or PAP2 cells (see Bautista et al., J. Biol. Chem. 269:23280-23285)) to one or more candidate therapeutic agents (this can be done in vivo or ex viv (for example, in cell culture)) and subsequently examining the level of OPN-b and OPN-c mRNA or protein expression in the cell. mRNA expression can be evaluated by Northern blot analysis, RNAse protection assays, or a PCR-based amplification assay (e.g., RT-PCR). Protein expression-can be evaluated by Western blot analysis or other antibody-based detection assay. Regardless of the exact method by which expression or activity is measured, appropriate controls can be set. For example, the expression or activity of OPN-b and OPN-c can be measured in the absence of the agent or in the presence of an agent that has been rendered inactive (by, for example, heat). Analogous assays can be performed to screen agents, including nucleic acid sequences, for their ability to increase the expression or activity of OPN-a in a cell (which may or may not express OPN-a naturally).

An agent that decreases the level of OPN-b and/or OPN-c mRNA or protein expression is an anti-osteopontin-b and/or anti-osteopontin-c therapeutic agent. Any, class of compounds, including those available in cDNA, synthetic, or chemical libraries can be tested. Alternatively, the agent can be found within a natural extract (e.g., a plant extract) or homogenate (or isolated therefrom).

Candidate therapeutic agents can also be evaluated in assays for OPN-b and OPN-c activities. For example, therapeutic agents can be evaluated by examining their effect on cellular proliferation or metastatic potential. An agent (e.g.. a small molecule) is an anti-osteopontin-b or anti-osteopontin-c therapeutic if it specifically inhibits OPN-b or OPN-c and/or subsequently inhibits the proliferation of a cell or the proliferative growth of a population of cells (e.g. a cell or cells in which growth control is lost) or the metastatic potential of a cell or cells within a population (these assays can include evaluation of the cell's ability to adhere to extracellular matrix or to invade non-cancerous tissue).

These assays, whether carried out in vivo or in cell culture, can also be carried out with cells that have been engineered to express or overexpress OPN-b or OPN-c (i.e; the expression level may be a natural level of expression or a heightened level of expression, which may provide a more sensitive assay condition). For example, the cell(s) used in the assays can be made to express a construct that encodes only an OPN-b transcript (or a biologically active fragment or other mutant thereof). Alternatively, the construct can express an OPN-c transcript (or a biologically active-fragment or other mutant thereof) and a heterologdus sequence that can be detected. For example, the construct can include a reporter or marker gene (i.e., any gene whose expression may be assayed such as luciferase, a green fluorescent protein (GFP or EGFP), α-glucoronidase (GUS), chloramphenicol transacetylase (CAT), or LacZ, which encodes β-galactosidase. In either event (whether a reporter or marker gene is included or not), one can examine OPN-b and/or OPN-c expression in the presence and absence of a potential therapeutic agent; an agent that decreases the expression or activity of OPN-b or OPN-c can be tested further in vivo or in vitro for an effect on cellular proliferation or some other indication of malignancy. The agent can interact with OPN-b or OPN-c mRNA or protein directly (by, for example, binding to the mRNA or protein) or indirectly (by binding to a cellular target that regulates OPN-b or OPN-c mRNA or protein expression, such as a transcription factor). For example, evidence suggests that amino acids 1-71 (FIG. 4) may be important for interactions with complement Factor H and Matrix Metalloproteinase-3 (MMP-3) (stromelysin-1) (Fedarko et al., J. Biol. Chem. 275: 16666-72, 2000; Agnihotri et al., J. Biol. Chem. 276: 28261-28267, 2001). The interaction between osteopontin and Factor H blocks the alternative complement pathway, providing one mechanism of tumor cells to escape from host humoral surveillance. The interaction of osteopontin with Factor H has been mapped to exon 4 (Jain et al., J. Biol. Chem. 277: 13700-8, 2002), which leads to the hypothesis that OPN-c, which is missing exon 4, is defective in binding to Factor H. Exon 5 is believed to be required for interaction with and activation of MMP-3, and thus OPN-b, which lacks exon 5, has lost the ability to activate MMP-3. It is not yet know how the osteopontin splice variants OPN-b and OPN-c facilitate malignancy, but the differential interactions of OPN-b and OPN-c with MMP-3 and Factor H may play certain roles in the cell transformation process. An agent that acts on MMP-3 and/or Factor H may play certain roles in the cell transformation process. An agent that acts on MMP-3 and/or Factor H in a way that compensates for the diminished association of either of these factors with osteopontin is a candidate for an anti-cancer therapeutic.

Anti-osteopontin-b and anti-osteopontin-c therapeutics can reduce the negative impact of OPN-b and OPN-c, respectively (on, for example, tumorigenesis), by shifting the equilibrium between OPN-b and OPN-c and each of the other osteoppntin isoforms. Thus; one can screen for, and subsequently formulate and administer to patients, agents that may not substantially inhibit the amount of OPN-b mRNA or protein in a cell, but rather reduce that amount relative to another isoform (e.g., OPN-a or OPN-c). Similarly, one can screen for, and subsequently formulate and administer to patients, agents that may not substantially inhibit the, amount of OPN-c mRNA or protein in a cell, but rather reduce that amount relative to, e.g., the OPN-a or OPN-b isoforms. One can detect or evaluate osteopontin isoforms in many ways. For example, one can transfect osteopontin-expressing cells with an engineered construct that expresses a luminescent fusion protein only if exons 4 and 5 are included. Exposure of the transfected cells to a potential therapeutic agent and a subsequent increase in luminance would indicate enhanced inclusion of exons 4 and exon 5 in the spliced mRNA. This result would suggest an increase in endogenous OPN-a levels relative to OPN-b and OPN-c isoforms. Accordingly, and while the invention is not limited to the use of agents that inhibit OPN-b or OPN-c expression or activity through any particular cellular mechanism, the therapeutic agents identified in such an assay can be administered to patients who have, or who are at risk for developing (initially or as a recurrent event) an osteopontin-b-expressing and/or osteopontin-c-expressing cancer.

Regardless of the parameter being measured (e.g., OPN-b and OPN-c expression or activity) or the agent being tested (e.g., an antisense oligonucleotide or small molecule), the conditions in which cells are exposed to test agents should allow the agent access to functional cells (e.g., the assay can be carried out at or near physiological temperatures and, in the event the cells are cultured, in the presence of art-recognized nutrients).

The invention also provides for methods to determine whether cells in a tumor or any suspicious growth are malignant or benign. The methods can be carried out by, for example, obtaining a sample of the tumor (or growth) and determining whether cells within the sample express OPN-b and/or OPN-c (any technique known in the art, including RT-PCR, Northern, and Western blot analyses can be used). Detecting the “b” or “c” isoform of osteopontin indicates a malignant tumor or growth (however, an absence of OPN-b or OPN-c does not necessarily indicate a non-malignant tumor).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated by reference in their entirety. The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is an illustration of the nucleotide sequence of OPN-a (SEQ ID NO: 1) (GenBank Accession number D28759). This isoform includes all six translated exons and has been identified in healthy tissue, as well as in benign and malignant tumors (the translation is represented by SEQ ID NO:2).

FIG. 2 is an illustration of the nucleotide sequence of OPN-b (SEQ ID NO:3) (GenBank Accession number D28760). This isoform is a splice variant that excludes exon 5 (the translation is represented by SEQ ID NO:4).

FIG. 3 is an illustration of the nucleotide sequence of OPN-c (SEQ ID NO:5) (GenBank Accession number D28761). This isoform is a splice variant that excludes exon 4 and has only been detected in malignant tumor cells (the translation is represented by SEQ ID NO:6).

FIG. 4 illustrates the structural characteristics of the osteopontin gene product. Top: The osteopontin gene has 6 translated exons. Sequences for splice variants of exons 4 (OPN-c, SEQ ID NO: 5) and 5 (OPN-b, SEQ ID NO: 3) are described in FIG. 3 and FIG. 2, respectively. Middle: The protein contains two primary domains: a central fragment contains the integrin binding sites, while the CD44 binding site lies on the C-terminal domain. Bottom: The integrin binding sites cover the sequence GRGDS (SEQ ID NO:7). The smallest integrin αᵥβ₃ binding peptide starts at AA71. Binding to β₁-containing integrins occurs through the non-canonical sequence SVVYGLR (SEQ ID NO:8), unless the β₁ chain is paired with α₄, in which case the binding site ranges from AA131 to AA144. The CD44v6 binding site covers the region from AA169 to AA220. Heparin-bridges between osteopontin and CD44v3 may be formed via the heparin binding sites on AA170 and 300. No known osteopontin functions have been mapped to the N-terminal domain (amino acids 1-71), which contains the alternatively spliced exons 4 and 5. The scheme is not drawn to scale.

FIG. 5A illustrates the expression of osteopontin splice variants in multiple tumor cell lines. RNA was extracted from cell lines, reverse transcribed, and used as template in PCR reactions. Primers for osteopontin amplified a 616 bp segment from the 5′ end of the transcript. No template (not shown) and GAPDH served as controls. In malignant breast cancer (MDA-MB-435) and lymphoma (HeLa), two osteopontin bands are amplified. In T-cells (Jurkat), one band is amplified. The double bands amplified from MDA-MB-435 cells and HeLa cells (and also Saos-2 cells, not shown) were cloned and sequenced.

FIG. 5B monitors osteopontin expression in breast tumor cell lines (top panel). RNA was extracted and RT-PCR was performed as described in FIG. 5A. Two bands are seen in the malignant cells (MDA-MB-435, 21MT1, 21MT2, MDA-MB-231). In benign cells (H16N2, MCF-7, ZR75) one band or no band is obtained. Normal breast epithelial cells express low or moderate amounts of standard osteopontin (76N, 70N, 7VNE, 3VN, 7VN; bottom panel). Breast epithelial cells immortalized with the HPV oncogene E6 (81E6, M2E6E7, 16E6P) express two transcripts of osteopontin (data not shown).

FIG. 5C is a Western blot analysis of osteopontin protein present in cell lysates. The number of transcripts detected by RT-PCR corresponds to the number of protein bands (arrows). The malignant cell line MB-435 produces two forms of osteopontin that are capable of being resolved by SDS-PAGE; only one osteopontin isoform is detected in the benign cell line MCF-7. The * indicates a likely cleavage product that is very commonly observed on Western blots for osteopontin.

FIG. 6 is a gel showing osteopontin mRNA splice variants amplified from transformed cell lines. The mRNA isoforms were amplified by RT-PCR.

FIG. 7 is a Coommassie blue stain of a protease digest of osteopontin substrate. Commercial MMP-3 (Chemicon) was activated by 0.25 mM APMA for 5 hours at 37° C. (see Example 2). Osteopontin (200 ng) was incubated with the active proteinase for 15 minutes at 37° C. After resolution on 10% SDS-PAGE and Coomrnassie blue staining, this yielded a faint cleavage band of around 45 kD (arrows). Osteopontin alone (lane 1) and MMP-3 alone (lane 6) served as controls. The synthetic peptide has a calculated molecular weight of 1.598 kDa and migrated with the dye front; no additional bands were observed after incubation of the peptide with MMP-3 in the absence of osteopontin.

FIG. 8 is an agarose gel stained with ethidium bromide to show the osteopontin isoforms cloned from MDA-MB-435 cells (see Example 3). Total RNA was extracted from the malignant breast tumor cell line MDA-MB-435. The osteopontin message was amplified with a primer pair that amplifies the coding region. The figure shows the results from mini-prepped DNA after TA cloning of the PCR products, which had shown a clear double band by agarose gel analysis. The bands in lanes 4 and 5 have been confirmed by sequencing to represent the wildtype “osteopontin-a” and its splice variant “osteopontin-b.”

### DETAILED DESCRIPTION

The following description sets out the compositions and methods of the present invention in more detail. As noted above, the methods can be carried out in vivo or in vitro (e.g., in cell culture) to detect tumors that express OPN-b and/or OPN-c, and in vivo to treat patients who are either suffer from or are at risk of developing a cancer, including a glioma (Saitoh et al., Lab. Invest. 72:55-63) or a malignancy of the colon, duodenum, stomach, breast, lung, prostate, bladder, ovary, thyroid, or pancreas (Brown et al., Am. J. Pathol. 145:610-623, 1994).

Generally, the methods of treating patients rely either on specific inhibition of OPN-b and/or OPN-c (e.g.,. methods in which OPN-b and/or OPN-c is inhibited to a greater extent than OPN-a) or those in which the amount of OPN-b and/or OPN-c expression or activity is reduced relative to that of OPN-a (thus, in some instances, the amount of OPN-b and/or OPN-c may not change at all). As such, the present methods are distinct from previous attempts to block osteopontin activity by blocking all forms of osteopontin, including the full length OPN-a (SEQ ID NO:1; see Feng et al., Clin. Exp. Metast. 13:453-462, 1995; Behrand. et al., Cancer Res. 54:832-837, 1994; Bautista et al., J Biol. Chem. 269:23280-23285, 1994; Thalmann et al., Clin. Cancer Res. 5:2271-2277, 1999; Helfrich et al., J. Bone Miner Res. 7:335-343, 1992; and Chambers et al., Cancer Res. 53:701-706, 1993; see also Saitoh et al., Lab. Invest. 72:55-63, 1995 and Kiefer et al., Nuc Acids Res 17:3306, 1989).

While the methods of the invention (particularly those directed to treatment or prophylaxis) are not limited to those achieved by any particular cellular mechanism, we suspect that by specifically inhibiting the activity of OPN-b and/or OPN-c, the host isoform, OPN-a, continues to function and, by doing so, exerts a cancer-fighting benefit on the cell and on the patient (the principle is the same when relative amounts of the three isoforms are adjusted).

Inhibiting OPN-b and OPN-c Translation

One way to inhibit OPN-b and OPN-c activity is to inhibit translation of the respective mRNAs. This can be accomplished using the small RNA endonucleases, called ribozymes, which cleave the phosphodiester bond of substrate RNA, thus specifically inhibiting the expression of target genes. Trans-acting hammerhead ribozymes contain a catalytic domain and flanking regions, which allow hybridization to the target sequence. Short stretches of RNA (possibly as low as 19 nucleotides) may suffice to generate catalytic activity.

Previous studies have indicated that osteopontin mRNA is amenable to targeting by ribozymes. Three hammerhead ribozymes designed to cleave three different regions of osteopontin mRNA reduced osteopontin expression in a subset of transformed cells. These cells were less tumorigeriic and metastatic (Feng et al., Clin. Exp. Metast. 13:453-462, 1995). The ribozymes described in Feng et al., cleave within the C-terminal half of the osteopontin mRNA, thereby targeting all three osteopontin isoforms (OPN-a, OPN-b and OPN-c). In contrast, the present invention provides for ribozymes that specifically inhibit expression of OPN-b or OPN-c mRNA but not of the full length (OPN-a) mRNA. For example, a mRNA sequence including the exon 4/exon 6 splice junction and flanking sequences can be used to select a catalytic RNA having a ribonuclease activity specific for OPN-b from a pool of RNA molecules. Similarly, a mRNA sequence including the exon 3/exon 5 splice junction and flanking sequences can be used to select a catalytic RNA having a specific ribonuclease activity specific for OPN-c (see, e.g., Bartel and Szostak, Science 261:1411-1418, 1993; see also Krol et al., Bio-Techniques 6:958-976, 1988).

Therapy with antisense oligonucleotides is also intended to prevent the translation of proteins associated with a particular disease state. Osteopontin antisense molecules have been expressed by stably transfecting cells with a mammalian expression vector containing an osteopontin cDNA fragment in an inverted orientation. In that case, the antisense RNA was capable of targeting all forms of OPN mRNA, and expression in metastatic ras-transformed NIH3T3 mouse fibroblasts caused reduced malignancy. Primary tumor growth rates in nude mice and in a chick embryo assay for metastasis were reduced or completely inhibited (Behrend et al., Cancer Res. 54: 832-837, 1994). Given the findings below, the present invention features methods for targeting OPN-b and OPN-c mRNA specifically. An antisense RNA, for example, that targets the exon 4/exon 6 splice junction will only inhibit translation of OPN-b, and an antisense RNA that targets the exon 3/exon 5 splice junction will only inhibit translation of OPN-c mRNA. In either of these cases, the full-length OPN-a continues to be translated. For example, the antisense oligonucleotide can be an RNA molecule (e.g., an 18-mer, a 19-mer, a 20-mer, a 21-mer or a 30-mer), complementary to the region including and flanking the splice junction of OPN-b or OPN-c (e.g., nucleotides 65-84, corresponding to OPN-b mRNA (SEQ ID NO: 2) or nucleotides 84-103, corresponding to OPN-c mRNA (SEQ ID NO: 3)).

While diagnostic and therapeutic methods are discussed further below, we note here that antisense nucleic acids can be administered to a subject according to protocols known in the art. For example, they can be injected into a particular tissue or generated in situ and, in either event, will hybridize with (or specifically bind to) the appropriate cellular osteopontin mRNA splice variant (OPN-b or OPN-c), thereby inhibiting expression of the encoded protein. Antisense nucleic acids can also be administered systemically and, if so, may be modified to target selected cells. For example, antisense nucleic acids can be linked to antibodies or other proteins (e.g., receptor ligands) that will specifically bind to cell surface receptors or other components associated with the target cell type. Similarly, the nucleic acids can include agents that facilitate their transport across the cell membrane (see, e.g.,: Letsinger et al., Proc. Natl. Acad. Sci. USA 86:6553-6556, 1989; Lemaitre et al., Proc. Natl. Acad. Sci. USA 84:648-652, 1987; and WO 88/09810) or the blood-brain barrier (see, e.g., WO 89/10134). In addition, nucleic acids can be modified with intercalating agents (Zon, Pharm. Res. 5:539-549, 1988). To achieve sufficient intracellular concentrations of antisense nucleic acids, one can express them in vectors having a strong promoter (e.g., a strong pol II or pol III promoter).

In other embodiments, antisense nucleic acids can be α-anomeric nucleic acids, which form specific double-stranded hybrids with complementary RNA in which, contrary to the usual β-units, the strands run parallel to each other (Gaultier et al., Nucleic Acids Res. 15:6625-6641, 1987). Alternatively, antisense nucleic acids can comprise a 2′-o-methylribonucleotide (Inoue et al., Nucleic Acids Res. 15:6131-6148, 1987) or a chimeric RNA-DNA analogue (Inoue et al., FEBS Lett. 215:327-330, 1987).

Targeting of OPN-b and OPN-c mRNAs by small inhibitory RNAs (siRNAs) is achieved by introducing a double-stranded RNA homologous to the sequence to be cleaved (e.g., the exon 4/exon 6 splice junction of OPN-b and the exon 3/exon 5 splice junction of OPN-c) (Tuschl et al., Genes Dev. 13:3191-3197, 1999). Methods of delivery are the same as or similar to those used for antisense molecules.

Inhibiting OPN-b and OPN-c Activity

Another approach to treating metastatic tumors is by inhibiting OPN-b and OPN-c proteins. Antibodies and synthetic peptides are the most common tools employed to inhibit protein activity (although agents other than antibodies and peptides can be used in the methods of the present invention). Various antibodies have been synthesized that recognize distinct epitopes of osteopontin, one of which targets exon 4, and thus fails to recognize OPN-c (Rittling et al., Biochem. Biophys. Res. Commun. 250:287-292, 1998; Kon et al., J. Cell Biochem. 77:487-498, 2000). Polyclonal antibodies-generated against osteopontin and isolated from human milk inhibited the growth stimulatory effect of osteopontin in human prostate carcinoma cancer cells (Thalmann et al., Clin. Cancer Res. 5:2271-2277, 1999). Previous investigations did not target osteopontin-b or osteopontin-c specifically, but instead inhibited all forms of osteopontin. Antibodies or peptides (or other agents) that specifically bind OPN-b or OPN-c allow for a targeted cancer therapy. To date, there are no antibodies specific to osteopontin-b or osteopontin-c. However, the amino acid sequence at the exon 4/exon 6 splice junction of OPN-b is suitable for antibody generation; the sequence KQNLLAPETLP (corresponding to AA51-61 of SEQ ID NO: 9) has a score of 1.091 in the program Antigenic, which predicts potentially antigenic regions of a protein sequence using the method of Kolaskar and Tongaonkar (FEBS Letters, 276:172-1.74, 1990). By a similar analysis, the amino acid sequence of OPN-c at the exon 3/exon 5 splice junction is not suitable for antibody generation, but the sequences ±10 amino acids around the splice site are unique for both osteopontin variants and therefore are, in principle, both suitable for the generation of specific antibodies. Thus, although antibodies specific for OPN-b and OPN-c do not yet exist, the potential to generate such antibodies is realistic, and these would be useful as specific anti-cancer agents in OPN-b and OPN-c expressing tumors.

Methods to identify compounds (unless specifically noted, the term “compound” may be used herein interchangeably with “test compound,” “agent,” “candidate therapeutic agent” and the like) that specifically inhibit OPN-b or OPN-c activity include cell-based assays of OPN-b and OPN-c expression or activity. These methods include culturing cells, for example mammalian cells, that express endogenous osteopontin, or an engineered osteopontin cassette, or both (ie., cells that naturally express OPN-b or OPN-c may also be transfected with an OPN-b or OPN-c expression vector, respectively), exposing the cells to a test compound (or a pool or group of test compounds), and analyzing OPN-b or OPN-c expression or activity. Expression can be detected by, for example, RT-PCR, Northern, and/or Western blot analysis. Activity can be examined by analyzing any OPN-b or OPN-c based event (e.g., inhibition of cellular proliferation). An assay for OPN-b expression would, include, for example, a decreased sensitivity to cleavage by MMP-3 (see above, and Example 2). An assay for OPN-c would include, for example, a decreased interaction with Factor H (see above). As noted above, the test compounds can include, but are not limited to, antisense oligonucleotides, ribozymes, siRNAs, small molecules, antibodies, or peptides. Such compounds can be collected or assembled into libraries for high throughput screening. Cassettes that express osteopontin-b or osteopontin-c for the purpose of identifying therapeutic agents may be stably transformed into cells or expressed from a constitutive or inducible promoter in a plasmid. Cassettes can include at least exons 3, 4, 5 and 6, and all or fragments of the intervening introns. For high throughput screening, the cassette may include a reporter gene, such as luciferase or GFP, that functions as an indicator for the inclusion of exons 4 and 5, and the subsequent negative effect on OPN-b and/or OPN-c expression.

Constructs. The invention also encompasses genetic constructs (e.g., plasmids, cosmids, and other vectors that transport nucleic acids) that include a nucleic acid of the invention, including, for example, a sequence that encodes the OPN-b or OPN-c protein or a fragment thereof (preferably, the fragment or other OPN-b or OPN-c mutant can be used to screen for agents that inhibit OPN-b or OPN-c expression or activity, respectively). The constructs may also contain sequences that encode an inhibitory agent, including, for example, an antisense RNA, ribozyme, siRNA, or peptide. The nucleic acids can be operably linked to a regulatory sequence (e.g., a promoter, enhancer, or other expression control sequence, such as a polyadenylation signal) that facilitates expression of the nucleic acid. The vector can replicate autonomously or integrate into a host genome, and can be a viral vector, such as a replication defective retrovirus, an adenovirus, or an adeno-associated virus.

Kits. The diagnostic and therapeutic methods to specifically target OPN-b and OPN-c isoforms can be assembled as kits. Accordingly, for diagnostic purposes, the invention features kits for detecting the presence of OPN-a, OPN-b and OPN-c mRNA transcripts or the proteins they encode in a biological sample. The kit can include a probe (e.g., a nucleic acid sequence or an antibody), a standard and, optionally, instructions for use. More specifically, antibody-based kits can include a first antibody (e.g., in solution or attached to a solid support) that specifically binds one of the osteopontin protein isoforms (OPN-a, OPN-b or OPN-c), and, optionally, a second, different antibody that specifically binds to the first antibody and is conjugated to a detectable agent. Oligonucleotide-based kits can include an oligonucleotide (e.g., a detectably labeled oligonucleotide) that hybridizes specifically to an OPN-a, OPN-b or OPN-c mRNA transcript under stringent conditions. For instance, the oligonucleotides can encode-a sequence that bridges the exon 4/exon 5 junction to indicate the presence of OPN-a. Alternatively, the oligonucleotides can encode a sequence that bridges the exon 4/exon 6 junction, or the exon 3/exon 5 junction to indicate the presence of OPN-b or OPN-c mRNA transcripts, respectively. The kit, optionally, can contain a mixture of the diagnostic oligonucleotides. The kits can be structured, for instance, for Northern blot analysis, or for in situ hybridizations.

One diagnostic kit also contains a triplet of oligonucleotides that can be used in RT-PCR analysis to amplify a nucleic acid sequence within any of SEQ ID NOs: 1, 3 or 5. One primer (e.g., an oligo(dT) primer, or a primer flanking a splice junction) is provided for reverse transcription of mRNA to-synthesize cDNA. A pair of primers is provided to PCR amplify the osteopontin splice variants. For example, the primers can hybridize to or around the relevant osteopontin splice junctions, within optional or common exons, or to the 5′ and 3′ UTSs flanking the coding region.

The kits can also include a buffering agent, a preservative, a protein-stabilizing agent, or a component necessary for detecting any included label (e.g., an enzyme or substrate). The kits can also contain a control sample or a series of control samples that can be assayed and compared to the test-sample contained. Each component of the kit can be enclosed within an individual container, and all of the various containers can be within a single package.

Patients Amenable to Treatment

Patients who are amenable to treatment by the therapeutic methods of the invention have, or are at risk for, a cancer. Examples of cancer types include, but are not limited to, carcinomas, sarcomas, leukemias and lymphomas. A metastatic tumor expressing OPN-b or OPN-c can arise from a multitude of primary tumor types, including but not limited to, those of the prostate, colon, lung, breast, intestine, stomach, bladder, ovary, thyroid, pancreas or liver. Patients having, or at risk for, a glioma are also candidates for anti-OPN-b and/or anti-OPN-c therapies. A human at risk for these cancers includes a healthy individual who has a family history of canter and an individual who has been treated (e.g., by surgery or with chemotherapies or radiation therapies) for a cancer that may recur.

Methods of Treatment

The present invention provides for both prophylactic and therapeutic methods of treating a subject at risk of (or susceptible to) a cancer or disease associated with OPN-b or OPN-c expression. “Treatment” encompasses the application or administration of a therapeutic agent to a patient, or to an isolated tissue or cell line (e.g., one obtained from the patient to be treated), with the purpose of curing or lessening the severity of the disease or a symptom associated with the disease. One advantage to the approach of targeting osteopontin splice variants to treat cancer is that, because the various osteopontin isoforms are secreted, the inhibitors do not necessarily need to penetrate the cell to be therapeutically effective.

As discussed, cancers associated (e.g., causally associated) with overexpression of OPN-b or OPN-c can be treated with techniques in which one inhibits the expression or activity of the OPN-b or OPN-c nucleic acid or gene product. For example, a compound (e.g., an agent identified using an assay described above) that exhibits negative modulatory activity with respect to OPN-b or OPN-c can be used to prevent and/or ameliorate a cancer, or one or more of the symptoms associated with it. The compound can be a peptide, phosphopeptide, small organic or inorganic molecule, or antibody (e.g., a polyclonal, monoclonal, humanized, anti-idiotypic, chimeric or single chain antibodies, and Fab, F(ab′)2 and Fab expression library fragments, scFV molecules, and epitope-binding fragments thereof).

Further, antisense, ribozyme and siRNA (see above) that inhibit expression of the OPN-b or OPN-c can also be used to reduce the level of OPN-b or OPN-c gene expression, respectively, thus effectively reducing the level of target gene activity. If necessary, to achieve a desirable level of gene expression, molecules that inhibit gene expression can be administered with nucleic acid molecules that encode and express OPN-b or OPN-c polypeptides exhibiting normal target gene activity.

Aptamer molecules (nucleic acid molecules having a tertiary structure that permits them to specifically bind to protein ligands; see, e.g., Osborne et al., Curr. Opin. Chem. Biol. 1:5-9, 1997 and Patel, Curr. Opin. Chem. Biol. 1:32-46, 1997) are also useful therapeutics. Since nucleic acid molecules can usually be more conveniently introduced into target cells than therapeutic proteins may be, aptamers offer a method by which protein activity can be specifically decreased without the introduction of drugs or other molecules that may have pluripotent effects.

Effective Dose:

Toxicity and therapeutic efficacy of the molecules disclosed in the invention (e.g., nucleic acids, polypeptides, ribozymes, antibodies etc.) and the compounds that modulate their expression or activity can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Polypeptides or other compounds that exhibit large therapeutic indices are preferred. While compounds that exhibit toxic side effects may be used, care should be taken to design a delivery system that targets such compounds to the site of affected tissue to minimize potential damage to uninfected cells and, thereby, reduce side effects.

Data obtained from the cell culture assays and further animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulations and Use: Pharmaceutical compositions for use in accordance with the present invention may be formulated in a conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The compounds may be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

The compounds may also be formulated in rectal compositions such as suppositories or retention enemas, for example, containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

The therapeutic compositions of the invention can also contain a carrier or eixcipient, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The nucleic acids, polypeptides, antibodies, or modulatory compounds of the invention can be administered by any standard route of administration. For example, administration can be parenteral, intravenous, subcutaneous, intramuscular, intracranial, intraorbital, opthalmic, intraventricular, intracapsular, intraspinal, intracisternal, intraperitoneal, transmucosal, or oral. The modulatory compound can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences.” It is expected that the preferred route of administration will be intravenous.

It is recognized that the pharmaceutical compositions and methods described herein can be used independently or in combination with one another. That is, subjects can be administered one or more of the pharmaceutical compositions, for example, pharmaceutical compositions comprising a nucleic acid molecule or protein of the invention or a modulator thereof, subjected to one or more of the therapeutic methods described herein, or both, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. By way of example, a subject afflicted with a disorder described herein can be simultaneously or sequentially administered both a cytotoxic agent which selectively kills aberrant cells and an antibody (e.g., an antibody of the invention) which can, in one embodiment, be conjugated or linked with a therapeutic agent, a cytotoxic agent, an imaging agent, or the like.

Detecting Malignant Cell Growth

The invention also provides methods to determine whether a tumor is malignant. By these methods, detection of OPN-b and OPN-c expression by common techniques known in the art, including RT-PCR, Northern or Western analysis, would provide complementary evidence that a tumor is malignant. The failure to detect OPN-b or OPN-c, however, should not be considered as sole proof that a tumor is not malignant.

### EXAMPLES

### Example 1

### Osteopontin-b and -c are Expressed in Malignant Tumor Cells, but Not in Benign Tumor Cells or Healthy Tissue

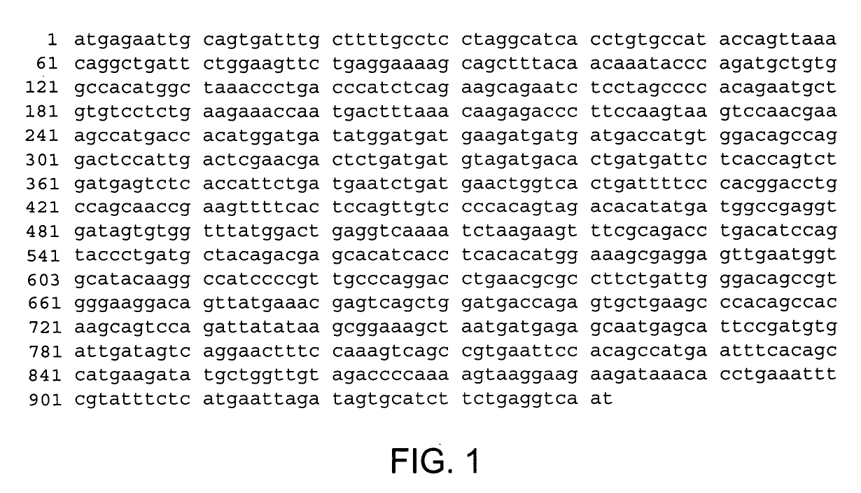

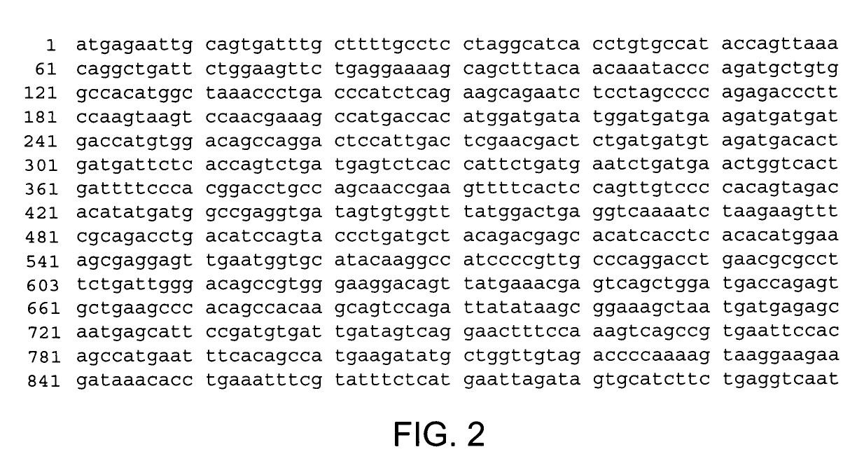

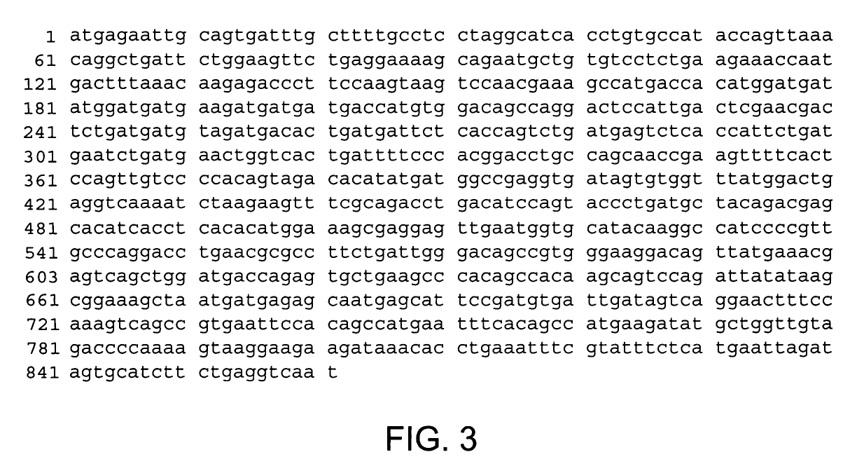

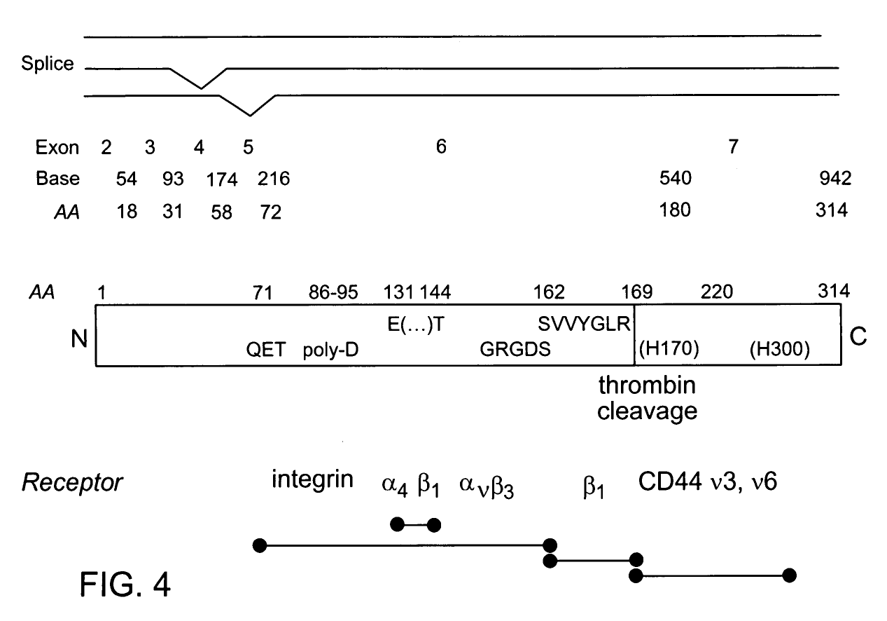

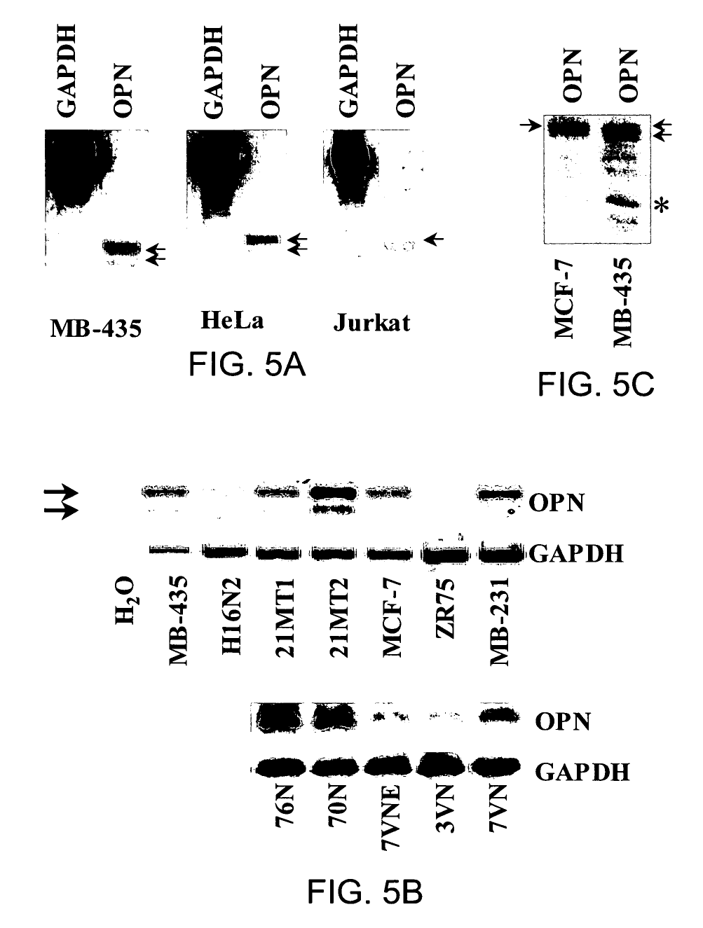

RNA was extracted from a variety of cell lines including the malignant cell lines MDA-MB-435, MDA-MB-231, 21MT1, 21MT2, Saos-2 and HeLa lymphoma cells; benign cell lines H16N2, MCF-7 and ZR75; the normal breast epithelial cell lines, 76N, 70N, 7VNE, 3VN and 7VN; breast epithelial cells immortalized with the APV oncogerie E6 including 81E6, M2E6E7 and 16E6P; and the T-cell line Jurkat. Osteopontin mRNA from several of these lines was analyzed by RT-PCR, cloning, and sequence analysis (see FIG. 5). Primers for osteopontin amplified a 616 bp segment from the 5′ end of the trancript. Reactions lacking a template and reactions in which GADPH was provided as the template served as controls. Alongside every other cell line, RNA from the malignant cell line MDA-MB435 cDNA was amplified to mark the two osteopontin bands that represent OPN-a and OPN-b. The two forms of osteopontin observed in the malignant cell lines MDA-MB435, HeLa, 21MT1, 21MT2 and Saos-2 were cloned and sequenced, and subsequently identified as OPN-a (SEQ ID NO: 1; FIG. 1) and OPN-b (SEQ ID NO:2; FIG. 2). In addition to the breast tumor cells described in the sequencing analyses (MDA-MB435, 21MT1 and 21MT2), gel mobility shift assays demonstrated that the breast tumor cell line MDA-MB-231 also expressed both OPN-a and the smaller variant OPN-b. Analysis by RT-PCR revealed the presence of various isoforms (FIG. 6).

The RT-PCR analysis of other cell lines revealed that in T-cells (Jurkat), only OPN-a is expressed (see FIG. 5). The normal breast epithelial cells 76N, 70N, 7VNE, 3VN and 7VN also expressed only low or moderate amounts of standard osteopontin. (OPN-a), and normal breast epithelial cells obtained from reduction mammoplasty (Liu et al., Cancer Res. 56:3371-9, 1996; Ratsch et al., Radiat. Res. 155 (1 Pt 2):143-150, 2001) also expressed only low or moderate amounts of OPN-a and no smaller transcripts.

### Example 2

### Osteopontin Cleavage by MMP-3 is Enhanced in the Presence of Osteopontin Exon 5 Peptide

Members of the matrix metalloproteinase family (MMP) are induced during injury and diseases in patterns overlapping with osteopontin expression (McCawley and Matrisian, Mol. Med. Today, 6: 149-156). MMP-3 cleaves at three sites (Gly166-Leu167, Ala201-Tyr202 and Asp210-Leu211) encoded by exons 6 and 7 of the human osteopontin protein, and MMP-cleaved OPN has demonstrated increased activity in promoting cell adhesion and migration compared with full-length OPN. In addition, the same receptors that interact with OPN mediate the interaction between MMP-3-cleaved OPN and tumor cells, suggesting that the cleaved form is an activated form of OPN, and that MMPs may function to regulate the activation of osteopontin protein (Agnihotri et al., Jour. Biol. Chem. 276:28261-28267, 2001).

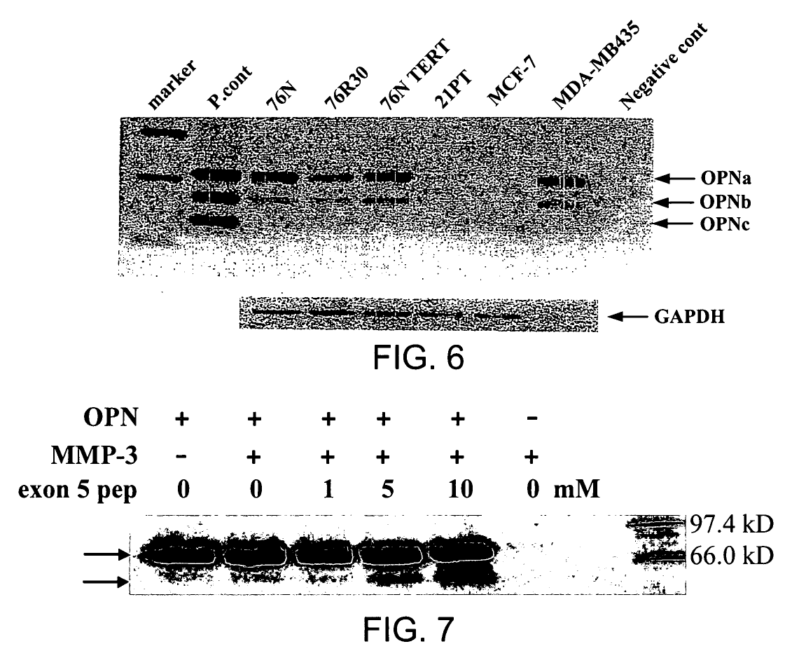

Evidence suggested that N-terminal domains may mediate the interaction between OPN and MMP-3 (Larry Fisher, NIDCR, NIH, “An Introduction to the SIBLING Family of Proteins,” 3^{rd} ICORP meeting, May 10-12, 2002). To test this hypothesis, commercial MMP-3 (Chemicon) was activated by 0.25 mM APMA for 5 hours at 37° C. 200 ng of osteopontin was incubated with the active proteinase for 15 min at 37° C. After resolution on, 10% SDS-PAGE and Coommassie blue staining, this yielded a faint cleavage band of around 45 kD (FIG. 7). The synthetic peptide has a calculated molecular weight of 1.598 kDa and migrated with the dye front; no additional bands were observed after incubation of the peptide with MMP-3 in the absence of osteopontin. Osteopontin cleavage was enhanced dose-dependently by the exon 5 peptide. Two additional experiments yielded similar results.

These results suggest that loss of exon 5 in OPN-b is sufficient to protect osteopontin from degradation by metalloproteinases. Tumor-derived OPN-b may aid invasiveness because of its increased half-life and resulting higher abundance. These results may also define the N-terminal MMP-interacting region as a third major functional domain on osteopontin, in addition to the central integrin-binding domain and the C-terminal CD44-binding domain.

### Example 3

### Osteopontin a and Osteopontin b are Cloned From MDA-MB-435 Cells

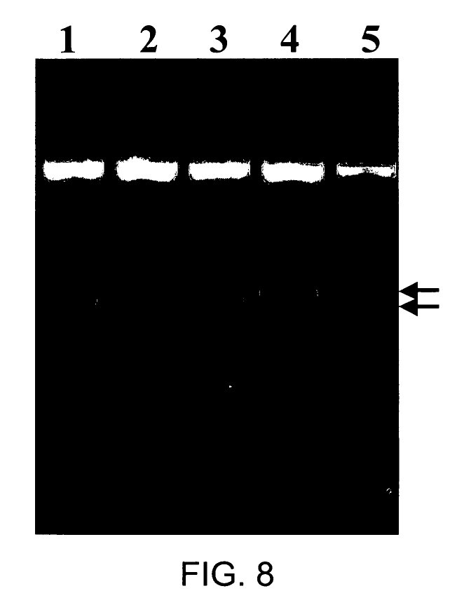

Total RNA was extracted from the malignant breast tumor cell line MDA-MB-435. The osteopontin message was reverse transcribed and then PCR amplified using a primer pair flanking the coding region. The PCR products ran as a clear double band on an ethidium bromide stained agarose gel, indicating the presence of at least two osteopontin isoforms. Both bands were cloned using the TA cloning method (Marchuk et al., Nucleic Acids Res. 19:1154, 1991), then plasmid DNA was miniprepped and restriction digested for analysis. The bands in lanes 4 and 5 (see FIG. 8) were sequenced and confirmed to represent the wildtype “osteopontin-a” and its splice variant “osteopontin-b.”

It is to be understood that, while the invention has been described in conjunction with the detailed description thereof, thee foregoing description is intended to illustrate and not limit the scope of the invention, which is defined by the scope of the appended claims. Other aspects, advantages, and modifications are within the scope of the following claims.

What is claimed:

1. A method of treating a patient who has a tumor, or who is at risk of developing a tumor, the method comprising administering to the patient an agent that inhibits the expression or activity of at least one of osteopontin-b or osteopontin-c (OPN-b or OPN-c).

2. The method of claim 1, wherein the patient has a carcinoma, a sarcoma, a leukemia, or a lymphoma.

3. The method of claim 1, wherein the patient has a tumor of the prostate gland, colon, lung, breast, stomach, bladder, ovary, thyroid gland, pancreas or liver.

4. The method of claim 1, wherein the patient has a glioma or wherein OPN-b has the sequence of SED ID NO: 3.

5. The method of claim 1, wherein OPN-c has the sequence of SED ID NO; 5.

6. The method of claim 1, wherein the agent inhibits the expression of at least one of OPN-b or OPN-c.

7. The method of claim 1, wherein the agent enhances inclusion of at least one of exon 4 or exon 5 in osteopontin mRNAs; degrades or inhibitis at least one of OPN-b or OPN-c mRNA; or degrades or inhibits at least one of OPN-b or OPN-c protein.

8. The method of claim 7, wherein the agent enhances inclusion of at least one of exon 4 or exon 5 in osteopontin mRNAs by modulating the splicing activity of OPN pre-mRNAs.

9. The method of claim 6, wherein the agent is (a) an oligonucleotide having a sequence antisense to at least one of OPN-b or OPN-c mRNA, but not anjisense to OPN-a mRNA, or (b) a ribozyme that specifically inhibits at least one of OPN-b or OPN-c expression.

10. The method of claim 9, wherein the ribozyme specifically targets a sequence at the exon 4/exon 6 splice junction of OPN-b mRNA or the exon 3/exon 5 splice junction of OPN-c mRNA.

11. The method of claim 9, wherein the oligonucleotide specifically targets a sequence at the exon 4/exon 6 splice junction of OPN-b mRNA or the exon 3/exon 5 splice junction of OPN-c mRNA.

12. The method of claim 6, wherein the agent is a small inhibitory RNA (siRNA) that specifically inhibits at least one of OPN-b or OPN-c expression.

13. The method of claim 12, wherein the siRNA is homologous to the exon, 4/exon 6 splice junction of OPN-b or the exon 3/exon 5 splice junction of OPN-c mRNA.

14. The method of claim 6, wherein the agent is an aptamer.

15. The method of claim 6, wherein the agent is an anti-OPN-b or anti-OPN-c antibody.

16. The method of claim 6, wherein the agent is a peptide or chemical compound.

17. The method of claim 1, wherein the agent is administered in conjunction with a chemotherapeutic compound, a radiation therapy, or a surgical procedure designed to excise the tumor.

18. A method for identifying an agent that inhibits the expression or activity of at least one of OPN-b or OPN-c, the method comprising:
a. providing a test compound,
b. administering the test compound to a cell, and
c. evaluating the level of OPN-B or OPN-c expression or activity, a decrease in expression or activity indicating that the test compound is an agent that inhibits the expression or activity of OPN-b or OPN-c.

19. The method of claim 18, wherein the test compound is an olgonucleotide having a sequence that is antisense to OPN-b or OPN-c niRNA, but not antisense to OPN-a mRNA; a ribozyme that specifically inhibits at least one of OPN-b or OPN-c expression; an siRNA that specifically inhibits OPN-c expression; or an aptamer, antibody, peptide or chemical compound that specifically inhibits at least one of OCPN-b or OPN-c expression.

20. The method of claim 19, wherein the oligonucleotide or ribozyme specifically targets a sequence at the exon 4/exon 6 splice junction of OPN-b mRNA or the exon 3/exon 5 splice junction of OPN-c mRNA.

21. The method of claim 20, wherein the siRNA is homologous to the exon4/exon 6 splice junction of OPN-b mRNA or the exon 3/exon 5 splice junction of OPN-c mRNA.

22. The method of claim 18, wherein the level of at least one of OPN-b or OPN-c expression is determined by RT-PCR, Northern blot analysis, RNAse protection assay, or Western blot analysis.

23. The method of claim 18, wherein the cell is a cell in culture.

24. The method of claim 18, wherein the cell is a cell in vivo.

25. The method of claim 18, wherein the cell naturally expresses at least one of OPN-b or OPN-c.

26. The method of claim 18 or claim 25, wherein the cell includes an exogenous sequence encoding at least one of OPN-b or OPN-c.

27. A method for determining whether a tumor, is malignant, the method comprising providing a sample of the tumor and determining whether cells within the tumor express at least one of OPN-b or OPN-c, expression of OPN-b or OPN-c being an indication that the tumor is malignant.

28. The method of claim 27, wherein expression of at least one of OPN-b or OPN-c is determined by RT-PCR, Northern blot analysis, RNAse protection assay, or Western blot analysis.
